(19) United States
(12) Patent Application Publication
(10) Pub. No.: US 2007/0031498 A1
(43) Pub. Date: Feb. 8, 2007

(54) GEL COMPOSITION FOR CELLULAR ADHESION INHIBITION

(75) Inventors: Xinhua Zong, San Jose, CA (US); Hungnan Lo, Shaker Heights, OH (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Wright Medical Technology, Inc.

(21) Appl. No.: 11/461,800

(22) Filed: Aug. 2, 2006

Related U.S. Application Data
(60) Provisional application No. 60/704,659, filed on Aug. 2, 2005.

(51) Int. Cl. A61K 31/737; A61K 31/727; A61K 9/14

(52) U.S. Cl. 424/486; 424/488; 514/54; 514/59; 514/56

(57) ABSTRACT

The invention includes compositions for inhibiting cellular adhesion, methods of preparation of such compositions, and methods for preventing cell adhesion at a surgical site comprising application of such compositions. The compositions generally comprise a cellular adhesion inhibitory agent, such as dextran sulfate, and a crosslinked hydrogel matrix, preferentially physically entrapping the adhesion inhibitory agent. The hydrogel matrix can include a first gel component, such as an electrophilically functionalized polyethylene glycol polymer, and at least one additional gel component, preferably nucleophilically functionalized, and preferentially selected from the group consisting of polyethylene glycol polymers, polypeptides, and polysaccharides. The compositions are useful for delivering the cellular adhesion inhibitory agent to a site in need of adhesion inhibition and providing either immediate or metered delivery of the inhibitory agent.

### CROSS-REFERENCE TO RELATED APPLICATIONS

The present application claims priority to U.S. Provisional Patent Application Ser. No. 60/704,659 filed Aug. 2, 2005, which is incorporated herein by reference in its entirety.

### FIELD OF THE INVENTION

The present invention relates to crosslinked hydrogel compositions comprising a cellular adhesive inhibitory agent. The hydrogel compositions are useful for delivery of the cellular adhesive inhibitory agent to a site in need of such inhibition, the hydrogels being preferably formulated for physically entrapping the cellular adhesive inhibitory agent, delivering the agent to the specified site, and releasing the agent, immediately or controllably, at the specified site for beneficial use.

### BACKGROUND

When injury or wounds occur in the human body, the body naturally reacts through mechanisms to repair the injury and close the wound. Many of these mechanisms are effective and beneficial. An example of such beneficial repair is epidermal regeneration in the presence of scratches, minor lacerations, and minor burns to the skin. Certain other cells in the body, such as hepatocytes, are also capable of regeneration, but it is generally limited to cases of minor injury and is most effective when healing conditions are optimal.

In situations involving major injury, such as surgery, the body's repair mechanism can result in the overgrowth of scar tissue. This can lead to complications ranging from minor, such as unsightly scars, to detrimental, such as surgical adhesions.

Surgical adhesions frequently occur following abdominal surgery and can generally be described as the binding of scarred tissue to adjacent tissue. The incidence of adhesions following abdominal surgery is cumulative with multiple surgeries, and female gynecological surgeries give a particularly high rate of adhesions. In one study, autopsy investigations indicated a 90% incidence of adhesions in patients with multiple surgeries, 70% incidence of adhesions in patients with a gynecologic surgery, and a 50% incidence of adhesions with appendectomy.

Surgical adhesions often result in serious post-surgical problems, including chronic pain, infertility, and bowel obstruction. Surgery is currently the only known treatment once the adhesions have formed. The widespread nature of the problem, as indicated by the above-noted study, was further confirmed by another study suggesting that a third of all abdominal surgery patients are readmitted to the hospital at least twice for further surgeries in relation to the adhesions.

Given the severity of the problems associated with cellular adhesion, various methods have been suggested to prevent formation of such adhesions. One such method is the application of a fine fabric barrier around the organs near a surgical site prior to completing the surgery.

U.S. Pat. No. 6,051,648 to Rhee et al. discloses the use of a cross-linked polyethylene glycol polymer for preventing the formation of adhesions following surgery. Rhee et al. generally discloses using the polymer coating as a protective barrier layer around the tissues. This activity is similar to the fabric barrier previously noted, functioning only as a physical barrier between adjacent tissues.

U.S. Pat. No. 5,605,938 to Roufa et al., which is incorporated herein by reference in its entirety, discloses the use of anionic polymers as inhibitors of scar formation, particularly surgical adhesions. Roufa et al. further disclose that the anionic polymers inhibit invasion of the cells associated with detrimental healing processes (i.e., inhibit fibroblast invasion), thus regulating the healing process and preventing fibrosis.

Roufa et al. disclose the use of adhesive proteins for anchoring the inhibitory anionic polymer at the site where inhibitory or regulatory activity is desired. The adhesive proteins are generally disclosed as including proteins containing a substantial amount of dihydroxyphenylalanine (DOPA) and hydroxyl-containing amino acid residues, such as fibrin-based products or fragments of polyphenolic adhesion protein from mussel, barnacle, or oyster.

It would, therefore, be useful to have further compositions incorporating effective cellular adhesion inhibitory agents for use as adhesion inhibiting agents, particularly compositions that facilitate easy, controllable delivery of the active component of the composition.

### SUMMARY OF THE INVENTION

The present invention provides cell anti-adhesive hydrogel matrix compositions comprising a cellular adhesion inhibitory agent and a polymeric delivery vehicle for controlled delivery of the inhibitory agent. Further provided are methods of preparation of cell anti-adhesive hydrogel matrix compositions and methods of preventing cell adhesion at a surgical site through use of such compositions.

According to one embodiment of the invention, there is provided a composition comprising 0.1 to 8 weight percent of a cellular adhesion inhibitory agent. Preferentially, the cellular adhesion inhibitory agent is an anionic polymer. In one preferred embodiment, the agent is selected from the group consisting of alginate, chondroitan sulfate, dermatan sulfate, dextran sulfate, hyaluronic acid, heparin, heparin sulfate, keratan sulfate, and pentosan polysulfate. The composition further comprises 92 to 99.9 weight percent of a crosslinked hydrogel matrix based upon the total weight of the composition. The crosslinked hydrogel matrix comprises a first hydrogel component comprising a polyethylene glycol polymer having at least one electrophilic group, and further comprises at least one additional hydrogel component having at least one nucleophilic group. Preferentially, the at least one additional hydrogel component is selected from the group consisting of polyethylene glycol polymers, polypeptides, and polysaccharides.

The cellular adhesion inhibitory agent can interact with the crosslinked hydrogel matrix according to various chemical and physical interactions. In one embodiment, the cellular adhesion inhibitory agent is physically entrapped in the crosslinked hydrogel matrix. In further embodiments, the cellular adhesion inhibitory agent can be chemically conjugated to at least one hydrogel component. In yet further embodiments, the adhesion inhibitory agent is chemically associated with at least one hydrogel component, such as through ionic interactions.

In another embodiment according to the present invention, there is provided a composition for inhibiting cellular adhesion comprising a cellular adhesion inhibitory agent and a crosslinked hydrogel matrix, wherein the hydrogel matrix comprises a first polyethylene glycol polymer having at least one electrophilic group and a second polyethylene glycol polymer having at least one nucleophilic group. Preferentially, the first and second polyethylene glycol polymers each have a molecular weight that is similar. For example, in one embodiment, each of the first and second polyethylene glycol polymers have a molecular weight of about 10,000 Da to about 20,000 Da. Additionally, it is preferable for the molar ratio of the first polyethylene glycol polymer to the second polyethylene glycol polymer to be about 1. Particularly preferred, according to this embodiment, is a composition wherein the cellular adhesion inhibitory agent is dextran sulfate and it is physically entrapped in the crosslinked hydrogel matrix.

In still another embodiment, the present invention provides a composition for inhibiting cellular adhesion comprising a cellular adhesion inhibitory agent and a crosslinked hydrogel matrix comprising a first polyethylene glycol polymer, a second polyethylene glycol polymer, and a polysaccharide. The first polyethylene glycol polymer includes one or more electrophilic groups, and the second polyethylene glycol polymer includes one or more nucleophilic groups. Further, the first polyethylene glycol polymer and the second polyethylene glycol polymer are covalently crosslinked. The polysaccharide component of the hydrogel matrix can be chemically or physically associated with at least one of the first and second polyethylene glycol polymer components. Preferentially, when the polysaccharide component is chemically associated, the polysaccharide is chemically conjugated to the first polyethylene glycol polymer. Further, preferentially, when the polysaccharide component is physically associated, the polysaccharide is physically entrapped in the covalently crosslinked first and second polyethylene glycol polymers. In one particularly preferred embodiment of the invention, the polysaccharide component includes chitosan. It is also preferred that the cellular adhesion inhibitory agent is dextran sulfate and is physically entrapped in the crosslinked hydrogel matrix.

According to another aspect of the present invention, there is provided a method for preparing a cell anti-adhesive crosslinked hydrogel matrix. In one embodiment according to this aspect of the invention, the method comprises the following steps: providing a first polyethylene glycol polymer having one or more electrophilic groups; mixing the first polyethylene glycol polymer with a solution containing at least one cellular adhesion inhibitory agent; and reacting the first polyethylene glycol polymer with a second polyethylene glycol polymer having one or more nucleophilic groups, thereby forming a crosslinked hydrogel matrix and physically entrapping the cellular adhesion inhibitory agent within the matrix. In one preferred embodiment, the cellular adhesion inhibitory agent includes an anionic polymer. Preferentially, the cellular adhesion inhibitory agent is selected from a group consisting of alginate, chondroitan sulfate, dermatan sulfate, dextran sulfate, hyaluronic acid, heparin, heparin sulfate, keratan sulfate, and pentosan polysulfate.

In another embodiment, the present invention provides a method for preparing a cell anti-adhesive crosslinked hydrogel matrix comprising providing a first gel component comprising a polyethylene glycol polymer having one or more electrophilic groups, mixing the first gel component with a solution of a cellular adhesion inhibitory agent, and reacting the first gel component with a second gel component having one or more nucleophilic groups, thereby forming a crosslinked hydrogel matrix and physically entrapping the cellular adhesion inhibitory agent within the hydrogel matrix. Preferably, the second gel component is selected from the group consisting of polyethylene glycol polymers, polypeptides, and polysaccharides and the cellular adhesion inhibitory agent is dextran sulfate.

In yet another embodiment of this aspect of the present invention, there is provided a method for preparing a cell anti-adhesive crosslinked hydrogel matrix comprising the following steps: providing a first gel component comprising a polyethylene glycol polymer having one or more electrophilic groups; mixing the first gel component with chitosan; providing a second gel component comprising a polyethylene glycol polymer having one or more nucleophilic groups; mixing the second gel component with a solution containing at least one cellular adhesion inhibitory agent; and reacting the second gel component with the first gel component to form a crosslinked hydrogel matrix and physically entrapping the cellular adhesion inhibitory agent within the hydrogel matrix. Preferentially, the cellular adhesion inhibitory agent is an anionic polymer. Further, preferentially, the cellular adhesion inhibitory agent is selected from the group consisting of alginate, chondroitan sulfate, dermatan sulfate, dextran sulfate, hyaluronic acid, heparin, heparin sulfate, keratan sulfate, and pentosan polysulfate. In one preferred embodiment, the chitosan is chemically conjugated to the first gel component. In another preferred embodiment, the chitosan is physically entrapped in the crosslinked hydrogel matrix formed by reacting the first and second gel components.

According to another aspect of the present invention, there are provided methods for preventing cell adhesion, such as cell adhesion at a surgical site. According to an embodiment of this aspect of the invention, the method comprises preparing a cell anti-adhesive cross-linked hydrogel matrix, and applying the hydrogel matrix to a surgical site. Preferentially, preparing the cell anti-adhesive cross-linked hydrogel matrix comprises providing a first gel component comprising a polyethylene glycol polymer having at least one electrophilic group, mixing the first polyethylene glycol polymer with a solution of a cellular adhesion inhibitory agent, such as dextran sulfate, and reacting the first gel component with a second gel component, thereby forming a crosslinked hydrogel matrix and physically entrapping the cellular adhesion inhibitory agent in the hydrogel matrix. In one embodiment, the second gel component is selected from a group consisting of polyethylene glycol polymers, polypeptides, and polysaccharides.

According to another embodiment of the invention, there is provided a method for preventing cell adhesion at a surgical site comprising providing a cell anti-adhesive crosslinked hydrogel matrix, and applying the hydrogel matrix to a surgical site. The cell anti-adhesive crosslinked hydrogel matrix comprises, according to one embodiment, a cellular adhesion inhibitory agent selected from the group consisting of alginate, chondroitan sulfate, dermatan sulfate, dextran sulfate, hyaluronic acid, heparin, heparin sulfate, keratan sulfate, and pentosan polysulfate. The hydrogel matrix further comprises a crosslinked hydrogel matrix comprising a first hydrogel component comprising a polyethylene glycol polymer having at least one electrophilic group, and at least one additional hydrogel component having at least one nucleophilic group. Preferentially, the at least one additional hydrogel component is selected from the group consisting of polyethylene glycol polymers, polypeptides, and polysaccharides.

### BRIEF DESCRIPTION OF THE DRAWINGS

FIG. 1 is a graphical representation of the effect of chitosan concentration in a hydrogel matrix according to the present invention on the release rate of dextran sulfate; and

FIG. 2 is a graphical representation of the ability of the inventive composition to prevent cellular adhesion in comparison to other treatments.

### DETAILED DESCRIPTION OF THE INVENTION

The present invention now will be described more fully hereinafter. However, this invention may be embodied in many different forms and should not be construed as limited to the embodiments set forth herein; rather, these embodiments are provided so that this disclosure will satisfy applicable legal requirements. As used in this specification and the claims, the singular forms “a,” “an,” and “the” include plural referents unless the context clearly dictates otherwise.

The present invention provides compositions containing a cell anti-adhesive component in immediate or controlled release form, methods of preparations of such compositions, and methods of preventing cellular adhesions through use of such compositions.

The compositions of the present invention generally comprise a crosslinked hydrogel matrix and at least one cellular adhesion inhibitory agent associated, either chemically or physically, with the crosslinked hydrogel matrix. The cellular adhesion inhibitory agent can be any agent effective for inhibiting adhesion of a biological material to another biological material or a non-biological material. Preferably, the cellular adhesion inhibitory agent is an agent effective for inhibiting fibrosis. Particularly useful as an anti-adhesive agent according to the present invention are biocompatible anionic polymers known to be effective for inhibiting scar formation, in particular surgical adhesion, and also known to be effective for inhibiting fibrosis in general. Such polymers are useful to inhibit fibroblast invasion, thus regulating the healing process and preventing fibrosis. The polymers are also useful for inhibiting glial cell invasion, bone growth, and neurite outgrowth.

Multiple biocompatible anionic polymers are known, and any of such polymers would be useful in the compositions of the present invention. For example, any of the following anionic polymers would be useful as a cellular adhesion inhibitory agent in the present compositions: alginate; chondroitan sulfate, dermatan sulfate, dextran sulfate, hyaluronic acid, heparin, heparin sulfate, keratan sulfate, and pentosan polysulfate. Further, any of the above-noted anionic polymers could be used alone or together, in any combination. Accordingly, in the compositions of the present invention, the cellular adhesion inhibitory agent can include one of the above anionic polymers. Alternatively, the cellular adhesion inhibitory agent can include two or more of the above anionic polymers. Further, the cellular adhesion inhibitory agent can include one or more of the above anionic polymers in combination with one or more additional agents known to be useful for inhibiting cellular adhesion. In one embodiment of the invention, the cellular adhesion inhibitory agent includes dextran sulfate. In another embodiment of the invention, the cellular adhesion inhibitory agent includes pentosan polysulfate. In another embodiment of the invention, the cellular adhesion inhibitory agent can include both dextran sulfate and pentosan polysulfate.

The inhibitory agent can further include disaccharides of one or more of the anionic polymers. Further, the inhibitory agent can include glycosaminoglycans and proteoglycans including one or more of the anionic polymers.

Anionic polymers for use in the present invention can be obtained from natural sources (e.g., proteoglycans), and can be used as found in nature or purified. Additionally, the anionic polymer can be prepared synthetically, such as through chemical derivatization. For example, the polyglucose polymer dextran can be treated by boiling in sulfuric acid and esterifying with chlorosulfonic acid to produce dextran sulfate (see, e.g., The Merck Index, 10^{th} Edition, 1983, No. 2915, page 427). Biocompatible anionic polymers are readily available from commercial sources.

The cellular adhesion inhibitory agent should be present in the compositions of the present invention in an amount effective to at least partially inhibit cellular adhesion. Accordingly, in one embodiment of the invention, the composition comprises about 0.01 to about 12 weight percent of the cellular adhesion inhibitory agent, based on the total weight of the composition. Preferably, the cellular adhesion inhibitory agent comprises about 0.05 to about 10 weight percent of the composition. More preferably, the cellular adhesion inhibitory agent comprises about 0.1 to about 8 weight percent of the composition. In one particular embodiment, the cellular adhesion inhibitory agent comprises about 0.5 to about 2 weight percent of the composition, based on the total weight of the composition.

Particularly useful compositions according to the present invention comprise a cellular adhesion inhibitory agent that includes dextran sulfate, which is a long chain glucose polymer having the structural formula as provided below in formula 1: 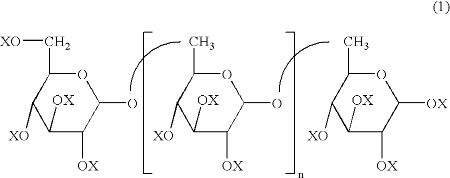
wherein X is hydrogen or sulfate (SO₃), and n is an integer between about 100 and about 10,000.

As seen in formula 1, the sulfur content of the dextran sulfate can vary. Sulfur content (i. e., relative number of sulfate groups present) can influence the effectiveness of the dextran sulfate as a cellular adhesion inhibitory agent as it is known that, in part, the effective anionic character of a polymer helps determine its inhibitory potential. Accordingly, in one embodiment of the invention, the dextran sulfate used in the present invention has a sulfur content of greater than 5 weight percent based upon the total weight of the dextran sulfate. Preferably, the dextran sulfate has a sulfur content of greater than 8 weight percent, more preferably, greater than 10 weight percent, based upon the total weight of the dextran sulfate. In another embodiment, the dextran sulfate has a sulfur content of greater than 12 weight percent based upon the total weight of the dextran sulfate. In yet another embodiment, the dextran sulfate has a sulfur content of greater than 15 weight percent based upon the total weight of the dextran sulfate.

As seen from formula 1 above, the molecular weight of dextran sulfate can vary based upon the value of n and the number of sulfate groups present. Preferably, the dextran sulfate used in the cellular adhesion inhibitory agent of the invention has an average molecular weight of about 40,000 to about 2,000,000 Da. In one embodiment, the dextran sulfate has a molecular weight of about 50,000 to about 1,000,000 Da. In another embodiment, the dextran sulfate has a molecular weight of about 75,000 to about 500,000 Da. Unless otherwise noted, molecular weight is expressed herein as weight average molecular weight (M_{w}), which is defined by formula 2 below

$${M}_{w} = \frac{\sum {n}_{i} {M}_{i}^{2}}{\sum {n}_{i} {M}_{i}} , \qquad ( 2 )$$

wherein nᵢ is the number of polymer molecules (or the number of moles of those molecules) having molecular weight Mᵢ.

The dextran sulfate used in preparing a composition for inhibiting cellular adhesion according to the invention is preferably in an aqueous solution. As used herein, a solution generally encompasses various aqueous mixtures of at least one solute and at least one solvent that would be apparent to one of skill in the art, including dispersions. Preferably, a dextran sulfate solution used in preparing a composition according to the invention has a concentration suitable for providing a final cellular adhesion inhibitory composition having a dextran sulfate concentration as provided above. For example, in one embodiment, the dextran sulfate solution has a concentration suitable for preparing a cellular adhesion inhibitory composition having dextran sulfate concentration of about 0.01 to about 12 weight percent. In one particular example, a dextran sulfate solution having a concentration of about 5 weight percent can be used to prepare a cellular adhesion inhibitory composition having a dextran sulfate concentration of about 2.5 weight percent, based on the overall weight of the solution.

In addition to a cellular adhesion inhibitory agent, as described above, the compositions of the present invention further comprise a crosslinked hydrogel matrix. The hydrogel matrix is particularly useful for facilitating a favorable release profile for the cellular adhesion inhibitory agent. As such, the hydrogel matrix can be formulated for delivery of the cellular adhesion inhibitory agent to a site wherein cellular adhesion inhibition is required so that such delivery can be immediate, delayed, or prolonged, as required for the specific use. For example, if inhibition of cellular adhesion is desirable only for a short time, the hydrogel matrix could be formulated such that substantially all of the cellular adhesion inhibitory agent could be released at the site of need shortly after delivery to the site. If inhibition of cellular adhesion is desirable for a prolonged period of time, the hydrogel matrix could formulated such that the release of the cellular adhesion inhibition agent is slower, but maintained over a longer period of time, with such time period being adjustable.

The crosslinked hydrogel matrix is present in the compositions of the present invention in an amount beneficial for achieving the above-stated functions. Accordingly, the crosslinked hydrogel matrix generally comprises about 88 to about 99.99 weight percent of the compositions of the present invention. Preferably, the hydrogel matrix comprises about 90 to about 99.95 weight percent, more preferably, about 92 to about 99.9 weight percent. In one specific embodiment, the crosslinked hydrogel matrix comprises about 98 to about 99.5 weight percent of the composition for inhibiting cellular adhesion, as provided by the present invention.

The crosslinked hydrogel matrix generally comprises a first hydrogel component and at least one additional hydrogel component. The first hydrogel component comprises a synthetic hydrophilic polymer. Preferentially, the first hydrogel component comprises a polyethylene glycol (PEG) polymer. As known in the art, PEG polymers are polymers according to the general structure shown below in formula 3
—CH₂CH₂O—(CH₂CH₂O)ₙ—CH₂CH₂— (3)
wherein n is an integer from about 10 to about 4,000. Any PEG polymer according to the above structure could be useful according to the invention. In one particular embodiment of the invention, n can be an integer from about 50 to about 3,000, more particularly about 100 to about 2,000, still more particularly about 200 to about 500. In one specific embodiment of the invention, n is an integer from about 250 to about 450, particularly about 300 to about 400.

PEG is a highly versatile polymer available in multiple forms, making it particularly useful according to the present invention. The PEG polymer, for example, can exist in its non-bound form as a linear polymer with terminal hydroxyl groups as shown below in formula 4
HO—CH₂CH₂O—(CH₂CH₂O)ₙ—CH₂CH₂—OH (4)
which can be abbreviated as HO-PEG-OH, wherein the PEG portion is understood to represent the structure provided above in formula 3.

Multi-arm or branched PEG polymers are also useful according to the present invention. Multi-arm PEG polymers generally have two or more PEG backbones extending from a non-reactive linking chain. For example, a 6-arm PEG polymer generally could be illustrated as shown below in formula 5. 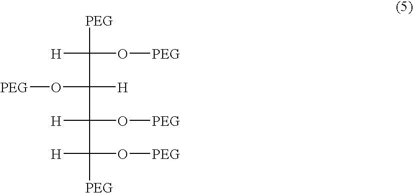
Similarly, for example, a 4-arm PEG polymer generally could be illustrated as shown below in formula 6. 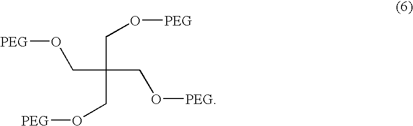
Such multi-arm PEG polymers as shown above in formulas 5 and 6 are readily available, such as from SunBio Corporation (Orinda, Calif.).

The at least one additional hydrogel component can be a synthetic or naturally occurring polymer and comprises a polymeric material selected from the group consisting of polyethylene glycol polymers, polypeptides, and polysaccharides. For example, the at least one additional hydrogel component can include a synthetic hydrophilic polymer, such as a synthetic PEG polymer substantially similar to the PEG polymer used as the first hydrogel component. Further, the at least one additional hydrogel component can include a natural or synthetic polypeptide. According to one embodiment, the polypeptide can include collagen, gelatin, poly(lysine), recombinant collagen, or recombinant gelatin. One particularly preferred polypeptide is collagen-derived gelatin. Similarly, the at least one additional hydrogel component can include a natural or synthetic polysaccharide. Examples of useful polysaccharides include chitosan and other amine-containing polysaccharides.

The first hydrogel component and the at least one additional hydrogel component are capable of chemically interacting, such as through covalent crosslinking, thereby forming a crosslinked hydrogel matrix. Preferentially, the hydrogel components are functionalized, the chemical interaction occurring between the functional groups on each hydrogel component. As used herein, the term “functionalized” is intended to mean that the respective hydrogel component includes at least one functional group (i.e., a group that is capable of reacting with another functional group to form a covalent bond). Such functional groups can be naturally occurring on the hydrogel component, or the hydrogel component can be chemically derivatized to include one or more functional groups. As used herein, the term “crosslinking” is intended to describe the attachment of two chains of polymer molecules by bridges composed of either an element, a group, or a compound that join certain carbon atoms of one polymer chain with certain polymer atoms of the other chain by primary chemical bonds. Typically, in a crosslinked system, the polymer chains are covalently attached along multiple points on each polymer backbone.

The first hydrogel component preferably includes at least one functional group that is an electrophilic group. Further preferably, the first hydrogel component can include multiple electrophilic groups. In one embodiment of the invention, the first hydrogel component includes 2 to 6 electrophilic groups. Exemplary electrophilic groups useful in the present invention include succinimidyl groups, aldehyde groups, benzotriazloe groups, and isocyanate groups. The at least one additional hydrogel component preferably includes at least one functional group that is a nucleophilic group. Again, the at least one additional hydrogel component can preferably include multiple nucleophilic groups. In one embodiment of the invention, the second hydrogel component includes 2 to 6 nucleophilic groups. Exemplary nucleophilic groups useful in the present invention include groups such as amine groups and thiol groups. As would be recognized by one of skill in the art, any electrophilic group and nucleophilic group that would be suitable for interacting with one another to form covalent crosslinking between the first hydrogel component and the at least one additional hydrogel component would be useful according to the present invention.

In one embodiment of the invention, the crosslinked hydrogel matrix includes PEG-succinimidyl glutarate and PEG-amine. Preferentially, the PEG-succinimidyl glutarate includes 4 or 6 succinimidyl groups and the PEG-amine includes 4 or 6 amine groups. As examples of the functionalized PEG polymers, a 6-arm PEG-amine polymer as could be used according to the present invention is provided below in formula 7. 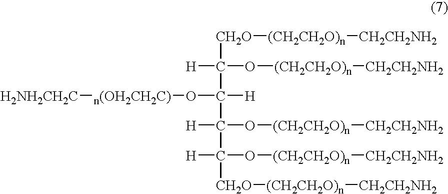
As a further example, a 4-arm PEG-succinimidyl glutarate polymer as could be used according to the present invention is provided below in formula 8.

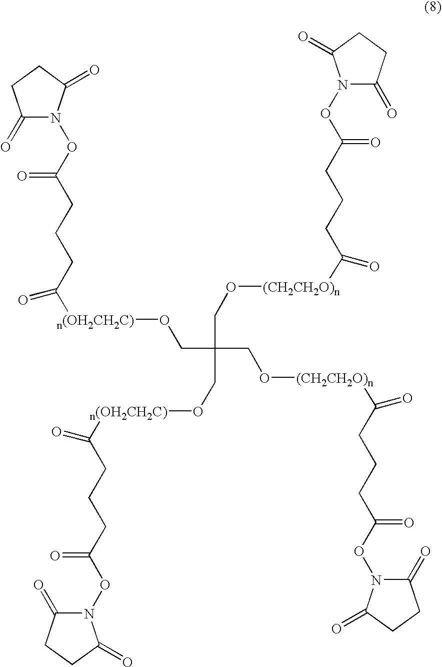

The compositions for inhibiting cellular adhesion as provided by the present invention generally comprise a cellular adhesion inhibitory agent and a crosslinked hydrogel matrix. The cellular adhesion inhibitory agent is associated with, and interacts with, the crosslinked hydrogel matrix in such a way that facilitates delivery of the cellular adhesion inhibitory agent by the crosslinked hydrogel matrix to a site, such as a surgical site, wherein cellular adhesion inhibition is beneficial. The association and interaction of the cellular adhesion inhibitory agent with the crosslinked hydrogel matrix can be either a chemical interaction, such as a chemical conjugation, or a physical interaction. Chemical conjugation, as used herein, refers to a chemical linkage formed by covalent bonding. Chemical conjugation is not to be confused with covalent crosslinking, wherein multiple covalent bonds are formed between polymer strands along the polymer backbones. Chemical conjugation, rather, is merely intended to describe the formation of one, or a few, covalent bonds. Wherein a structure bonded by covalent crosslinking is tightly bound with a more cohesive structure, a group that is chemically conjugated to another group can be more easily de-bonded.

Chemical interaction of the cellular adhesion inhibitory agent with the crosslinked hydrogel matrix encourages formation of a composition wherein the adhesion inhibitory agent is more slowly released at a site of delivery, the release being dependent upon the degradation rate of the crosslinked hydrogel matrix. According to this embodiment of the invention, the adhesion inhibitory agent is released from the hydrogel matrix at a rate that is dependent upon degradation of the hydrogel matrix into smaller components through natural body processes. As would be recognized by one of skill in the art, the degradation rate of the crosslinked hydrogel matrix may be varied according to the different functional groups of the hydrogel components realizing that the bonds between the functional groups will be degraded at different rates. Further, degradation could be controlled through inclusion of specific degradable groups in the backbone structure of the hydrogel matrix components.

In one preferred embodiment of the invention, the cellular adhesion inhibitory agent is associated with the crosslinked hydrogel matrix by a physical interaction, wherein the cellular adhesion inhibitory agent is physically entrapped in the crosslinked hydrogel matrix. This embodiment is particularly preferred in that the release rate of the cellular adhesion inhibitory agent is quicker in comparison to compositions wherein the cellular adhesion inhibitory agent chemically interacts with the hydrogel matrix. Physical entrapment of the cellular adhesion inhibitory agent in the hydrogel matrix allows for release of the inhibitory agent by diffusing out of the matrix. Accordingly, the composition can be formulated such that the inhibitory agent readily diffuses out of the hydrogel matrix providing a quick, high concentration of the inhibitory agent at the site where adhesion inhibition is needed. However, the diffusion of the inhibitory agent out of the hydrogel matrix can be slowed or delayed, such as through inclusion in the hydrogel matrix of a component capable of charge interactions with the cellular adhesion inhibitory agent.

According to the above general description of the invention, one particular embodiment of the invention provides a composition for inhibiting cellular adhesion comprising dextran sulfate physically entrapped in a crosslinked hydrogel matrix. The crosslinked hydrogel matrix comprises a first PEG polymer having at least one electrophilic functional group, and a second PEG polymer having at least one nucleophilic group. As described above in relation to Formula 3, the molecular weight of the PEG polymers can vary depending upon the value of n. PEG polymers of varying molecular weight can be used according to the invention. In one embodiment of the invention, it is useful for the first PEG polymer and the second PEG polymer to each be of a similar molecular weight. For example, in one embodiment, the first PEG polymer and the second PEG polymer can each have a molecular weight of about 10,000 Da to about 20,000 Da. Of course, the molecular weight of the PEG polymers used in the invention is not intended to be limited to such range.

It can also be useful according to the invention for the first PEG polymer and the second PEG polymer to be present in amounts such that the molar ratio of electrophilic groups to nucleophilic groups is greater than or equal to one. Having a molar excess of electrophilic groups can be beneficial, particularly when a third hydrogel component, such as a polysaccharide, is present as the excess electrophilic groups can facilitate chemical conjugation of the polysaccharide. In embodiments comprising only two hydrogel components, such as a first PEG polymer and a second PEG polymer, it is beneficial for the molar ratio of electrophilic groups to nucleophilic groups to be about 1:1.

As previously noted, the crosslinked hydrogel matrix as used in the composition of the present invention can generally include a first hydrogel component and at least one additional hydrogel component. In one embodiment of the invention, the hydrogel matrix includes a first hydrogel component and at least two additional hydrogel components. With the presence of at least two additional hydrogel components, the crosslinked hydrogel matrix can form between various hydrogel components. For example, covalent crosslinking can occur between three or more hydrogel components. Accordingly, the crosslinked hydrogel matrix can include two electrophilically functionalized hydrogel components crosslinked to one nucleophilically functionalized hydrogel component. Alternately, the crosslinked hydrogel matrix can include one electrophilically functionalized hydrogel component crosslinked to two nucleophilically functionalized hydrogel components.

Further, according to this embodiment, covalent crosslinking can occur between two hydrogel components, while one of the crosslinked hydrogel components further chemically interacts with at least a third hydrogel component, such as through chemical conjugation. As such, the at least third hydrogel component is chemically associated with at least one of the first and second hydrogel components, but the at least third hydrogel component is non-participatory in the covalent crosslinking of the hydrogel matrix.

Still further, according to this embodiment, covalent crosslinking can occur between two hydrogel components while at least a third hydrogel component is physically associated with the crosslinked hydrogel matrix. Such a physical interaction, depending upon the exact chemical nature of the specific hydrogel components, would be expected to include attractive forces between the hydrogel components, such as hydrogen bonding, van der waals forces, and charge interactions.

According to one particularly preferred embodiment of the invention, a composition for inhibiting cellular adhesion is provided wherein the composition comprises dextran sulfate and a crosslinked hydrogel matrix comprising a first PEG polymer having one or more electrophilic group, a second PEG polymer having one or more nucleophilic group and being covalently crosslinked to the first PEG polymer, and a polysaccharide. The polysaccharide can be chemically associated with the first PEG polymer or second PEG polymer. Further, the polysaccharide can be physically associated with at least one of the first PEG polymer or second PEG polymer. Preferentially, the dextran sulfate is physically entrapped in the crosslinked hydrogel matrix formed by the first PEG polymer and the second PEG polymer.

The polysaccharide component of the crosslinked hydrogel matrix, when it is physically associated with the first PEG polymer and/or the second PEG polymer, does not directly participate in formation of the crosslinked hydrogel matrix. In other words, while covalent crosslinking occurs between the first electrophilically functionalized PEG polymer and the second nucleophilically functionalized PEG polymer, there is no covalent crosslinking between the polysaccharide component and the first PEG polymer or between the polysaccharide component and the second PEG polymer. In embodiments wherein the polysaccharide is physically associated with at least one of the first PEG polymer or second PEG polymer, the polysaccharide is physically entrapped in the hydrogel matrix formed of the two PEG polymers, such entrapment possibly supplemented by additional force interactions, such as described above.

As mentioned above, the polysaccharide can alternatively be chemically associated with one of the PEG polymers. For example, the polysaccharide can be chemically conjugated to a PEG polymer (i.e., can form covalent bonds at only one or a few points on the PEG polymer without significantly affecting the crosslinking between the PEG polymers). Crosslinking of the polysaccharide component can be substantially avoided through use of a nucleophilically functionalized PEG polymer (such as PEG-amine) that has a bonding reactivity that is greater than the reactivity of the reactive groups on the polysaccharide.

The inclusion of the polysaccharide component into the crosslinked hydrogel matrix of the composition is beneficial in regulating the release rate of the cellular adhesion inhibitory agent. Preferably, the polysaccharide used in preparing the crosslinked hydrogel matrix includes charged groups capable of ionically interacting with the charged groups on the cellular adhesion inhibitory agent. These ionic interactions function to prevent or slow diffusion of the cellular adhesion inhibitory agent out of the crosslinked hydrogel matrix. Accordingly, the release of the cellular adhesion inhibitory agent at the site in need of adhesion inhibition can be metered through adjustment of the concentration of the polysaccharide component of the crosslinked hydrogel matrix.

Polysaccharides particularly useful for inclusion in the hydrogel matrix for controlling the release rate of the cellular adhesion inhibitory agent include amine group-containing polysaccharides. In one embodiment of the invention, the polysaccharide used in the crosslinked hydrogel matrix is chitosan. Chitosan is a product of the deacetylation of chitin, which is the polymer of N-acetyl-D-glucosamine. Chitin, which is formed of the repeating unit shown below in formula 9, includes an acetamido group at the 2′ carbon. When the acetamido group is removed, such as through treatment with a strong base, such as sodium hydroxide, the resultant polymer is referred to a chitosan, the repeating unit of which is shown below in formula 10. 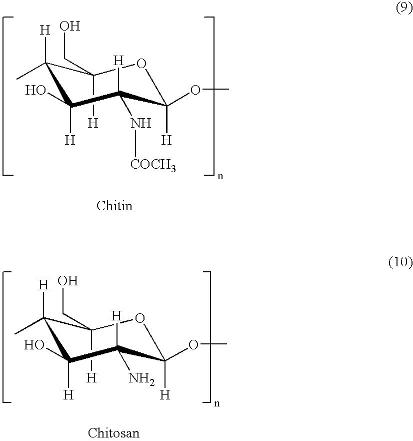
Generally, only a percentage of the acetyl groups are removed during deacetylation of the chitin (although, chitosan can exist in a completely deacetylated state). Accordingly, chitosan is generally referred to by its degree of deacetylation (e.g., chitosan is commonly available as 80-90% deacetylated chitin). In the present invention, chitosan that is at least about 70% deacetylated is preferred. Even more preferable, the chitosan is at least about 80% deacetylated. Most preferably, the chitosan is at least about 90% deacetylated.

The chitosan used in the present invention can be in a dry form (e.g., powdered form) or can be in a solution. When in solution form, the chitosan solution concentration can be such as would be useful for preparing a final composition according to the invention having an overall chitosan content of about 0.01 to about 15 weight percent based upon the total weight of the composition. For example, in one embodiment of the invention, a chitosan solution having a concentration of about 2 weight percent can be used to prepare a cellular adhesion inhibitory composition having a final chitosan concentration of about 1 weight percent, based on the overall weight of the composition.

The crosslinked hydrogel matrix of the invention can include a polypeptide component. Polypeptides, as used herein, can encompass tissue-derived or synthetic polypeptides, including collagen and collagen-derived polypeptides, such as gelatins, as well as recombinant collagen and gelatin or poly amino acids, such as poly(lysine). Preferentially, the polypeptides comprise sequences of amino acids having groups capable of interacting with other groups. Polypeptides used according to the present invention preferably have an average molecular weight of about 5,000 to about 1,000,000 Da, more preferably about 10,000 to about 500,000 Da, most preferably about 15,000 to about 100,000 Da.

One polypeptide particularly useful in the hydrogel matrix of the present invention is a gelatin, such as collagen derived gelatin. Gelatin is a denatured form of collagen obtained through partial hydrolysis of collagen.

One particular embodiment of the present invention provides a composition comprising dextran sulfate and a crosslinked hydrogel matrix, wherein the hydrogel matrix comprises a first PEG polymer having 4 or 6 succinimidyl groups, a second PEG polymer having 4 or 6 amine groups, and chitosan. The chitosan can be chemically conjugated to the first PEG polymer. Alternately, the chitosan can be physically entrapped in the crosslinked hydrogel matrix formed by the first PEG polymer and the second PEG polymer.

When chitosan is present in the crosslinked hydrogel matrix of the composition, the chitosan preferentially comprises about 0.01 to about 15 weight percent of the composition based upon the total weight of the composition. In one embodiment, the chitosan comprises about 0.02 to about 10 weight percent based upon the total weight of the composition. Preferably, the chitosan comprises about 0.05 to about 8 weight percent based upon the total weight of the composition.

Preferentially, the polysaccharide component, such as chitosan, is present in an amount useful for affecting the release rate of the cellular adhesion inhibitory agent. For example, in the embodiment wherein the composition comprises dextran sulfate physically entrapped in a hydrogel matrix comprising a first PEG polymer crosslinked with a second PEG polymer and chitosan physically associated therewith, it is preferred for the chitosan and dextran sulfate to both be present in an amount such that the molar ratio of chitosan to dextran sulfate is about 1:1. When the molar ratio is close to 1, ionic interactions between the dextran sulfate and the chitosan are capable of retaining the dextran sulfate within the hydrogel matrix, thereby providing a delayed release profile. To ensure delayed release, it is possible, according to the invention, for the chitosan to be present in molar excess. The invention, however, also encompasses embodiments wherein it may be beneficial for a portion of the dextran sulfate to be retained in the hydrogel matrix only by physical entrapment without ionic interaction with the chitosan. In such an embodiment, a portion of the dextran sulfate would be available for immediate release from the hydrogel matrix, while the remaining dextran sulfate could be released more slowly due to the ionic interactions with the chitosan. Accordingly, the invention also encompasses embodiments wherein the molar ratio of chitosan to dextran sulfate is from about 1:2 to about 3:1. Preferably, the molar ratio of chitosan to dextran sulfate is about 1:1 to about 2: 1, most preferably about 1:1.

According to another aspect of the present invention, there is also provided a method for preparing a cell anti-adhesive crosslinked hydrogel matrix. The method generally comprises providing a first gel component having one or more electrophilic groups, mixing the first gel component with a cellular adhesion inhibitory agent, such as dextran sulfate, and reacting the first gel component with a second component having one or more nucleophilic groups to form a crosslinked hydrogel matrix, thereby physically entrapping the cellular adhesion inhibitory agent within the matrix. Preferentially, the first gel component is an electrophilically functionalized PEG polymer. The second gel component, according to one embodiment, is a PEG polymer. In another embodiment, the second gel component is a polypeptide, as previously described, such as gelatin. In yet another embodiment, the second gel component is a polysaccharide, as previously described, such as chitosan. In further embodiments, the method can comprise chemically or physically associating further hydrogel components.

In one particular embodiment according to this aspect of the invention, the method initially comprises providing a first PEG polymer having one or more electrophilic groups and combining the first PEG polymer with at least one cellular adhesion inhibitory agent to prepare a mixture of the first PEG polymer and the at least one cellular adhesion inhibitory agent. As previously noted, the cellular adhesion inhibitory agents used according to the present invention are preferably anionic polymers, such as dextran sulfate. Preferentially, the cellular adhesion inhibitory agent is in solution. As such, the cellular adhesion inhibitory agent and an electrophilically functionalized PEG polymer, such as PEG-succinimidyl glutarate, are capable of physical admixture with little or no risk of unfavorable interactions, such as ionic interactions, that could destabilize the mixture. Conversely, admixture of an anionic cellular adhesion inhibitory agent with a nucleophilically functionalized polymer, such as PEG-amine, would be expected to be unstable in a buffered solution as the ionic interactions of the anionic cellular adhesion inhibitory agent and the cationic PEG-amine could lead to at least partial precipitation over a range of concentrations of the components. By pre-mixing the adhesion inhibitory agent with the electrophilically functionalized PEG polymer prior to introduction of a nucleophilically functionalized polymer, these undesirable ionic interactions are avoided.

Accordingly, in one embodiment of the invention, the method further comprises reacting the first PEG polymer with a second polymer having one or more nucleophilic groups, thereby forming a crosslinked hydrogel matrix and physically entrapping the cellular adhesion inhibitory agent within the hydrogel matrix.

Preferentially, the method for preparing a cell anti-adhesive crosslinked hydrogel matrix as provided in the present invention is effective for forming an “instant hydrogel”. The methods of the invention are preferentially capable of being carried out in vivo (i.e., formation of the hydrogel matrix is at the site of delivery of the cellular adhesion inhibitory agent). Alternately, the hydrogel of the invention can be prepared shortly before application. Accordingly, it is beneficial for the gel components to be capable of mixture at the time of use and thereby form a crosslinked hydrogel matrix useful for delivery of the cellular adhesion inhibitory agent within seconds or minutes of the mixing of the hydrogel components. Accordingly, as used herein, an “instant hydrogel” is a hydrogel wherein the gelled state is achieved within about 5 minutes of beginning the step of reacting the components to form the hydrogel matrix. Preferably, in the methods of the present invention, the reacting step of the methods requires a time of less than about 2 minutes. More preferably, the reacting step requires a time of less than about 60 seconds, most preferably less than about 30 seconds.

Since preparation of the composition of the invention can take place in different environments, it is preferable that the reacting step be capable of occurring over a range of temperatures. The reaction preferably occurs at a temperature ranging from room temperature (about 20° C.) to a slightly elevated body temperature (about 40° C.).

In light of the above, it is possible, according to the invention, to provide the components of the cell anti-adhesive crosslinked hydrogel matrix in pre-metered preparations for mixing and reacting at the time of use, such as by a physician or surgeon shortly before or at the time of delivery of the hydrogel matrix to the site in need of cellular adhesion inhibition. For example, the matrix components could be provided as three solutions: 1) a first PEG polymer solution; 2) a cellular adhesion inhibitory agent solution; and 3) a second PEG polymer solution. At the time of use, the physician or surgeon could mix solution 1 with solution 2 and then incorporate solution 3. The reaction would then proceed, and within about 30 seconds to about 5 minutes, the cell anti-adhesive crosslinked hydrogel matrix would be prepared and ready for use. Furthermore, the matrix components could be mixed and immediately applied to the site in need of cellular adhesion inhibition. The gel could then form at the site of application.

Similar to the above method, solutions 1 and 2 could be premixed into a single solution. Accordingly, the components as provided could comprise two solutions: 1) a mixture of a first PEG polymer and a cellular adhesion inhibitory agent; and 2) a second PEG polymer. For use, the physician or surgeon would need only to combine the two solutions to react the first PEG polymer and the second PEG polymer, thereby forming a crosslinked hydrogel matrix and physically entrapping the cellular adhesion inhibitory agent with the matrix. As before, the matrix would be ready for use once the reaction had occurred, which would be within about 30 second to about 5 minutes.

According to another aspect of the invention, there is provided a method for preparing a cell anti-adhesive crosslinked hydrogel matrix specifically incorporating a polysaccharide, such as chitosan, into the hydrogel matrix. The method comprises the following steps: 1) providing a first gel component; 2) mixing the first gel component with chitosan; 3) providing a second gel component; 4) combining the second gel component with a solution containing at least one cellular adhesion inhibitory agent; and 5) reacting the second gel component with the first gel component to form a crosslinked hydrogel matrix, physically entrapping the cellular adhesion inhibitory agent within the matrix. In this method, steps 3) and 4) could be carried out before steps 1) and 2) to produce the same result. In a particularly preferred embodiment, temporary charge interactions between the cellular adhesion inhibitory agent and the chitosan result when the crosslinked hydrogel matrix is produced.

In one particular embodiment of the invention, the first gel component is a PEG polymer having one or more electrophilic groups, the second gel component is a PEG polymer having one or more nucleophilic groups, and the cellular adhesion inhibitory agent is dextran sulfate.

While the compositions of the invention have been described herein in terms of solutions, it is further possible for the composition to be prepared as a non-hydrated mixture of the composition components capable of hydration at the time of use. For example, each component of the composition of the invention could be provided as a powder. The individual powders could be mixed together in the amounts necessary such that a later addition of a specified volume of liquid, such as water or a buffer solution, would provide a hydrated composition according to the invention ready for use. Alternately, the powdered composition could be hydrated to form a hydrogel in vivo. For example, an amount of the pre-mixed, non-hydrated composition could be placed directly in the body at an area where cellular adhesion inhibition is desired. Once placed, the powder could be hydrated through addition of an external fluid, or the hydration could be by natural body fluids alone, thereby forming a hydrogel with an adhesion inhibitory agent therein.

Providing the composition in a non-hydrated form is particularly useful in that the composition can be pre-mixed in metered amounts and stored for later use. Further, the pre-mixing can take place at a site different from the site of intended use, and the pre-mixed composition can be stored for extended time without adversely affecting the composition. Providing the composition in non-hydrated form also increases ease of use. For example, a powdered composition could be provided in various formulations (e.g., immediate release or delayed release) and in various amounts such that at the point of use, the only necessary preparation step is adding a predetermined volume of fluid to hydrate the composition. In some embodiments, even that step is unnecessary, as the powdered composition could be placed directly at the site of use and hydrated in vivo.

The composition of the invention readily lends itself to the non-hydrated form described above as the various components of the composition are generally readily available in non-hydrated, or powdered, form. Other methods of providing the composition in non-hydrated form, however, are also encompassed by the invention. For example, the composition of the invention could be prepared in hydrated form to exact specifications and then dehydrated by commonly used dehydration methods, such as freeze drying. The dehydrated composition could then be stored for later use. Such a method is beneficial, as the dehydrated composition could be formed by various methods for later use. For example, the dehydrated composition could be formed into sheets of various sizes that could be placed at a surgical site and re-hydrated in vivo. Further, the dehydrated composition could be ground into particles or formed into various other useful shapes.

In yet another aspect of the invention, there is provided a method for preventing cell adhesion at a surgical site. In one embodiment of the invention, the method comprises preparing a cell anti-adhesive crosslinked hydrogel matrix and applying the hydrogel matrix to a surgical site. Accordingly, in this embodiment, the method would encompass preparation of the hydrogel matrix as previously discussed. For example, the preparation could comprise providing a first gel component, mixing the first gel component with dextran sulfate, and reacting the first gel component with a second gel component. Furthermore, the present method would be expected to encompass any of the various methods as disclosed herein, as well as methods that may be inherent in the preparation of any of the compositions as disclosed herein. In particular, the present method would encompass the on-site preparation of the hydrogel matrix as previously described, wherein the gel components could be provided in separate solutions and be mixed immediately prior to use. Further, the method would encompass embodiments wherein the composition is provided in a non-hydrated form.

In another embodiment of this aspect of the invention, the method for preventing cell adhesion at a surgical site comprises providing a cell anti-adhesive crosslinked hydrogel matrix and applying the hydrogel matrix to a surgical site. In this embodiment, the method would be expected to encompass preparation of the hydrogel matrix in advance of the use thereof and then providing the previously made hydrogel for application to the surgical site. This method would also encompass preparation of the hydrogel matrix shortly before use and then delivery of the prepared hydrogel for application to the surgical site. Further, this method would also encompass use of a composition of the invention provided in non-hydrated form. Accordingly, any cellular adhesion inhibiting composition according to the present invention, when provided for application to a surgical site, would be encompassed by the present method. For example, the present invention would encompass providing a cell anti-adhesive crosslinked hydrogel matrix comprising a cellular adhesion inhibitory agent and a crosslinked hydrogel matrix wherein the crosslinked hydrogel matrix includes a first hydrogel component comprising a PEG polymer having at least one electrophilic group and at least one second hydrogel component having at least one nucleophilic group, and applying the hydrogel matrix to a surgical site.

Further embodiments of the present invention are more distinctly described according to the following experimental examples.

### Experimental

The present invention is more fully illustrated by the following examples, which are set forth to illustrate the present invention and are not to be construed as limiting.

### EXAMPLE 1

### Preparation of Adhesion Inhibitory Composition with Two Hydrogel Matrix Components

50 mg of dextran sulfate was dissolved in 1 ml of phosphate buffer solution (PBS) (1M, pH 7.4). Next, 0.1 g of 6-arm PEG-succinimidyl glutarate was added to the solution. Separately, 0.1 g of 4-arm PEG-amine was dissolved in 1 ml of PBS. The two solutions were combined to react the two PEG components. A hydrogel formed within about 30 to 60 seconds. The formed gel was a PEG/PEG hydrogel matrix with dextran sulfate physically entrapped therein.

### EXAMPLE 2

### Preparation of Adhesion Inhibitory Composition with Three Hydrogel Matrix Components

50 mg of dextran sulfate was dissolved in 1 mg of PBS. Next, 0.1 g of 4-arm PEG-amine was added to the solution. Separately, 20 mg of chitosan was dissolved in 1 ml of PBS and 0.1 g of 6-arm PEG-succinimidyl glutarate was added to the chitosan solution. The two solutions were combined to react the two PEG components. A hydrogel formed within about 30 to 60 seconds. The formed gel was a PEG/PEG hydrogel matrix with chitosan chemically conjugated to one PEG component and with dextran sulfate physically entrapped within the gel.

### EXAMPLE 3

### Non-Hydrated Formulations

Preparation of hydrogels having the compositions provided in Examples 1 and 2 can also be prepared using a non-hydrated mixture of the gel components. For a two-component hydrogel composition, 50 mg dextran sulfate, 0.1 g 6-arm PEG-succinimidyl glutarate, and 0.1 g 4-arm PEG-amine (all in powdered form) are mixed together, to provide a, preferentially, homogeneous mixture. The hydrogel of Example 1 can then be prepared by adding 2 ml of PBS to the above mixture.

For a three-component hydrogel composition, 50 mg dextran sulfate, 0. 1 g 4-arm PEG-amine, 20 mg chitosan, and 0.1 g 6-arm PEG-succinimidyl glutarate (all in powdered form) are mixed together to form a, preferentially, homogeneous mixture. The hydrogel of Example 2 can then be prepared by adding 2 ml of PBS to the mixture.

### EXAMPLE 4

### Sustained Release of Dextran Sulfate from the Hydrogel Matrix

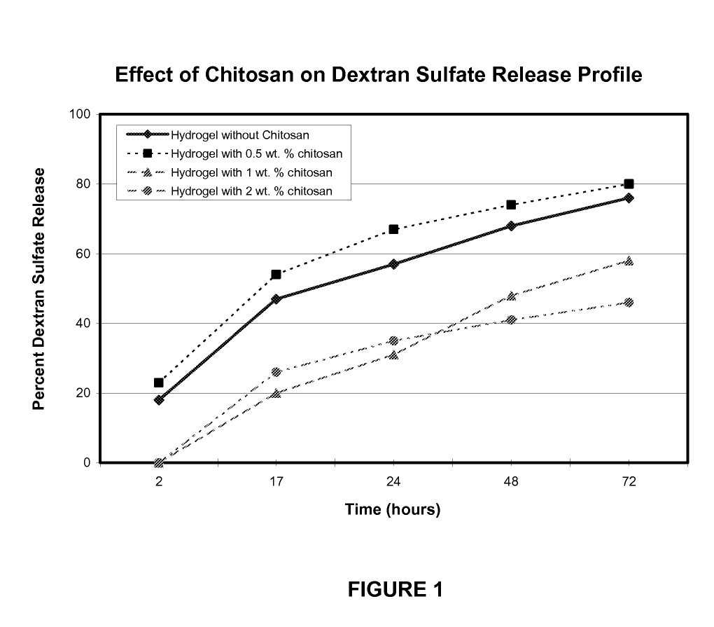

Four hydrogel compositions with dextran sulfate entrapped therein were prepared according to the procedure of Example 2. The amount of chitosan in samples 1-4 were 0 mg (0 wt. %), 10 mg (0.5 wt. %), 20 mg (1 wt. %), and 40 mg (2 wt. %), respectively. Each of the pre-weighed hydrogels were put into separate vials containing 30 ml PBS, and the vials were incubated in a water bath at 37° C. The release rate of dextran sulfate from each individual hydrogel was measured by HPLC at various time intervals over three days. The comparison of release profiles based on the amount of chitosan present in the hydrogel is provided in FIG. 1.

### EXAMPLE 5

### Use of Composition for Preventing Adhesion

Laminectomies were performed on multiple rabbits. In each rabbit, the laminectomies were at three separate sites from approximately L1 through L6. Individual incisions were used for each of the three sites to provide total separation of the three sites on each rabbit. The laminectomies measured approximately 10 mm by 5 mm and were allowed to remain open to allow for total hemostasis. In each rabbit, one site was treated with the composition of the invention prepared according to Example 1, a second site was treated with a gelatin/dextran sulfate composition, and a third site was only irrigated with saline. The order of application was randomized within each subject, but the levels receiving each treatment were rotated according to a pre-determined schedule in order to avoid bias. The wounds were then closed.

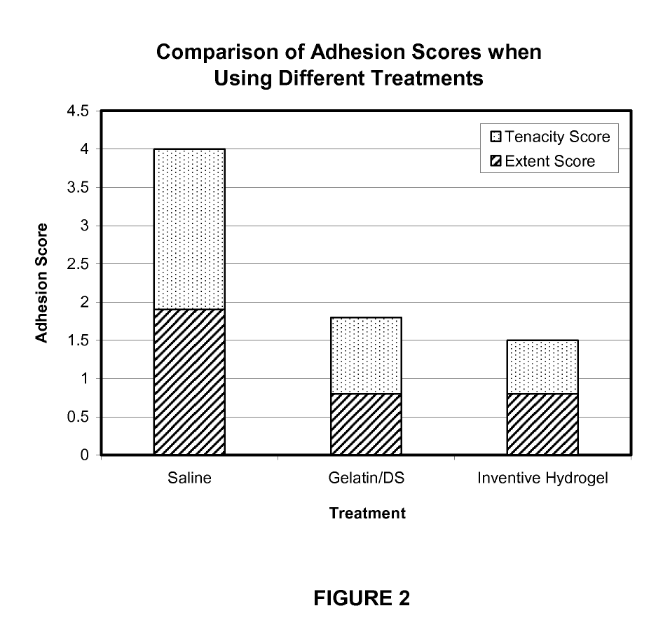

Approximately four weeks post-surgery, the subject were euthanized and the laminectomy sites exposed by careful dissection for evaluation. At each site, surgical adhesions were evaluated to determine the extent of surgical adhesion and, separately, the tenacity of the adhesions present. The extent of adhesion was rated on a scale of 0-3, with 0 indicating no adhesion and 3 indicating extensive adhesions. Similarly, the tenacity of the adhesions present was rated on a scale of 0-3, with 0 indicating little or no tenacity and 3 indicating highly tenacious adhesions. The overall adhesion score is the sum of the extent score and the tenacity score. The results of the study are provided in FIG. 2.

As can be seen in FIG. 2, both dextran sulfate compositions provide better adhesion scores than irrigation with saline. Further, the composition of the present invention outperformed the gelatin/dextran sulfate composition, particularly reducing the tenacity of the adhesions.

Many modifications and other embodiments of the inventions set forth herein will come to mind to one skilled in the art to which these inventions pertain having the benefit of the teachings presented in the foregoing description. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed is:

1. A composition for inhibiting cellular adhesion comprising:
0.1 to 8 weight percent of a cellular adhesion inhibitory agent based upon the total weight of the composition, the inhibitory agent being selected from the group consisting of alginate, chondroitan sulfate, dermatan sulfate, dextran sulfate, hyaluronic acid, heparin, heparin sulfate, keratan sulfate, and pentosan polysulfate; and
92 to 99.9 weight percent of a crosslinked hydrogel matrix based upon the total weight of the composition, the crosslinked hydrogel matrix comprising:
a first hydrogel component comprising a polyethylene glycol polymer having at least one electrophilic group; and
at least one additional hydrogel component having at least one nucleophilic group, the at least one additional hydrogel component being selected from the group consisting of polyethylene glycol polymers, polypeptides, and polysaccharides.

2. The composition according to claim 1, wherein the cellular adhesion inhibitory agent is physically entrapped in the crosslinked hydrogel matrix.

3. The composition according to claim 1, wherein the cellular adhesion inhibitory agent includes dextran sulfate.

4. The composition according to claim 3, wherein the dextran sulfate is present at 0.5 to 2 weight percent based on the total weight of the composition.

5. The composition according to claim 1, wherein the first polyethylene glycol polymer has a molecular weight of 10,000 Da to 20,000 Da.

6. The composition according to claim 1, wherein the first polyethylene glycol polymer comprises at least one succinimidyl group.

7. The composition according to claim 1, wherein the first polyethylene glycol polymer comprises 4 or 6 succinimidyl groups.

8. The composition according to claim 1, wherein the at least one additional hydrogel component includes a second polyethylene glycol polymer.

9. The composition according to claim 8, wherein the molar ratio of the first polyethylene glycol polymer to the second polyethylene glycol polymer is greater than or equal to 1.

10. The composition according to claim 8, wherein the second polyethylene glycol polymer has a molecular weight of 10,000 Da to 20,000 Da.

11. The composition according to claim 8, wherein the second polyethylene glycol polymer comprises at least one amine group.

12. The composition according to claim 11, wherein the second polyethylene glycol polymer comprises 4 or 6 amine groups.

13. The composition according to claim 1, wherein the at least one additional hydrogel component includes a polysaccharide.

14. The composition according to claim 13, wherein the at least one additional hydrogel component includes chitosan.

15. The composition according to claim 1, wherein the at least one additional hydrogel component includes a polypeptide.

16. The composition according to claim 16, wherein the at least one additional hydrogel component includes collagen.

17. The composition according to claim 15, wherein the at least one additional hydrogel component includes gelatin.

18. The composition according to claim 8, wherein the second polyethylene glycol polymer is covalently crosslinked to the first polyethylene glycol polymer, and wherein the at least one additional hydrogel component further includes a polysaccharide associated with at least one of the first polyethylene glycol polymer and the second polyethylene glycol polymer.

19. The composition according to claim 18, wherein the polysaccharide includes chitosan.

20. The composition according to claim 18, wherein the associated polysaccharide is chemically conjugated to the first polyethylene glycol polymer.

21. The composition according to claim 18, wherein the associated polysaccharide is physically entrapped in the covalently crosslinked first and second polyethylene glycol polymers.

22. The composition according to claim 1, wherein the crosslinked hydrogel matrix, in addition to the first hydrogel component, comprises at least two additional hydrogel components, at least one of the at least two additional hydrogel components having at least one nucleophilic group.

23. The composition according to claim 1, wherein the composition is in a non-hydrated form.

24. A method for preparing a cell anti-adhesive crosslinked hydrogel matrix comprising:
providing a first gel component comprising a polyethylene glycol polymer having one or more electrophilic groups;
combining the first gel component with at least one cellular adhesion inhibitory agent selected from the group consisting of alginate, chondroitan sulfate, dermatan sulfate, dextran sulfate, hyaluronic acid, heparin, heparin sulfate, keratan sulfate, and pentosan polysulfate; and
reacting the first gel component with a second gel component having one or more nucleophilic groups, the second gel component being selected from the group consisting of polyethylene glycol polymers, polypeptides, and polysaccharides, thereby forming a crosslinked hydrogel matrix and physically entrapping the at least one cellular adhesion inhibitory agent within the matrix.

25. The method according to claim 24, wherein the cellular adhesion inhibitory agent includes dextran sulfate.

26. The method according to claim 24, wherein said reacting step requires a time of less than 60 seconds.

27. The method according to claim 24, wherein the second gel component is a polyethylene glycol polymer.

28. The method according to claim 24, wherein the second gel component is a gelatin.

29. The method according to claim 24, wherein the second gel component is chitosan.

30. The method according to claim 24, further comprising dehydrating the cell anti-adhesive crosslinked hydrogel matrix for later use.

31. A method for preparing a cell anti-adhesive crosslinked hydrogel matrix comprising:
providing a first gel component comprising a polyethylene glycol polymer having one or more electrophilic groups;
mixing the first gel component with chitosan;
providing a second gel component comprising a polyethylene glycol polymer having one or more nucleophilic groups;
combining the second gel component with at least one cellular adhesion inhibitory agent selected from the group consisting of alginate, chondroitan sulfate, dermatan sulfate, dextran sulfate, hyaluronic acid, heparin, heparin sulfate, keratan sulfate, and pentosan sulfate; and
reacting the second gel component with the first gel component to form a crosslinked hydrogel matrix, physically entrapping the cellular adhesion inhibitory agent within the matrix.

32. The method according to claim 31, wherein the cellular adhesion inhibitory agent includes dextran sulfate.

33. The method according to claim 31, wherein said step of reacting the second gel component with the first gel component to form a crosslinked hydrogel matrix requires a time of less than 60 seconds.

34. The method according to claim 31, wherein said step of mixing the first gel component with chitosan comprises chemically conjugating the first gel component with the chitosan.

35. The method according to claim 31, wherein said step of mixing the first gel component with chitosan comprises physically entrapping the chitosan in the first gel component.

36. The method according to claim 31, further comprising dehydrating the cell anti-adhesive crosslinked hydrogel matrix for later use.

37. A method for preparing a cell anti-adhesive crosslinked hydrogel matrix comprising: combining (a) at least one cellular adhesion inhibitory agent selected from the group consisting of alginate, chondroitan sulfate, dermatan sulfate, dextran sulfate, hyaluronic acid, heparin, heparin sulfate, keratan sulfate, and pentosan sulfate,
(b) a first polyethylene glycol polymer having at least one electrophilic group,
(c) a second polyethylene glycol polymer having at least one nucleophilic group, and
(d) optionally, a polysaccharide, to form a cell anti-adhesive combination;
wherein each of the at least one cellular adhesion inhibitory agent, first polyethylene glycol polymer, second polyethylene glycol polymer and optional polysaccharide are in a non-hydrated form; and
hydrating the cell anti-adhesive combination to form a cell anti-adhesive crosslinked hydrogel matrix.

38. A method for preventing cell adhesion at a surgical site comprising:
(a) providing a cell anti-adhesive crosslinked hydrogel matrix, wherein the hydrogel matrix comprises:
(i) 0.1 to 8 weight percent of a cellular adhesion inhibitory agent selected from the group consisting of alginate, chondroitan sulfate, dermatan sulfate, dextran sulfate, hyaluronic acid, heparin, heparin sulfate, keratan sulfate, and pentosan polysulfate; and
(ii) 92 to 99.9 weight percent of a crosslinked hydrogel matrix based upon the total weight of the composition, the crosslinked hydrogel matrix comprising:
a first hydrogel component comprising a polyethylene glycol polymer having at least one electrophilic group; and
at least one additional hydrogel component having at least one nucleophilic group, the at least one additional hydrogel component being selected from the group consisting of polyethylene glycol polymers, polypeptides, and polysaccharides; and
(b) applying the cell anti-adhesive crosslinked hydrogel matrix to a surgical site where cell adhesion prevention is desired.

39. The method according to claim 38, wherein said providing step comprises preparing the cell anti-adhesive crosslinked hydrogel matrix, wherein said preparation comprises:
(i) providing a first gel component comprising a polyethylene glycol polymer having at least one electrophilic group;
(ii) mixing the first polyethylene glycol polymer with a solution of dextran sulfate having a concentration of 0.1 to 8 weight percent; and
(iii) reacting the first gel component with a second gel component having one or more nucleophilic groups, the second gel component being selected from the group consisting of polyethylene glycol polymers, polypeptides, and polysaccharides, thereby forming a crosslinked hydrogel matrix and physically entrapping the dextran sulfate.

40. The method according to claim 39, wherein the cell anti-adhesive crosslinked hydrogel matrix is in a non-hydrated form.

41. The method according to claim 40, further comprising re-hydrating the dehydrated cell anti-adhesive crosslinked hydrogel matrix.
